(19) United States
(12) Patent Application Publication
(10) Pub. No.: US 2013/0210027 A1
(43) Pub. Date: Aug. 15, 2013

(54) TEST SYSTEM AND METHOD

(71) Applicants: Anja Boisen, Birkerod (DK); Filippo Giacomo Bosco, Copenhagen Ø (DK); En-Te Hwu, Taipei City (TW)

(72) Inventors: Anja Boisen, Birkerod (DK); Filippo Giacomo Bosco, Copenhagen Ø (DK); En-Te Hwu, Taipei City (TW)

(73) Assignee: DANMARKS TEKNISKE UNIVERSITET, Lyngby (DK)

(21) Appl. No.: 13/825,108

(22) PCT Filed: Sep. 21, 2011
(86) PCT No.: PCT/DK11/50356

§ 371 Date: May 3, 2013

(30) Foreign Application Priority Data
Sep. 21, 2010 (DK) PA 2010 00849

(51) Int. Cl. G01N 33/53

(52) U.S. Cl. CPC G01N 33/53
USPC 435/7.1; 422/69; 435/287.2; 436/501

(57) ABSTRACT

The present invention relates to an apparatus for detecting compounds, the apparatus having a device defining a disk-shaped geometry, the device having a centre, a plurality of fluid channels each comprising a fluid inlet positioned at a first distance from the centre and a fluid channel end at a second distance from the centre, the second distance being larger than the first distance, one or more sensors arranged at each fluid channel, wherein the sensors each comprise at least one optical detectable member, the test apparatus further comprising one or more optical sensing devices arranged for sensing the at least one optical detectable member of the one or more sensors, and a rotation device adapted for rotating the device so that the sensors pass over the one or more optical sensing devices. Further the present invention relates to a method for determining compounds comprising providing an apparatus for detecting compounds having a device defining a disk-shaped geometry, the device having a centre, a plurality of fluid channels each comprising a fluid inlet positioned at a first distance from the centre and a fluid channel end at a second distance from the centre, the second distance being larger than the first distance, one or more sensors arranged at each fluid channel, wherein the sensors each comprise at least one optical detectable member, the test apparatus further comprising one or more optical sensing devices arranged for sensing the at least one optical detectable member of the one or more sensors, and a rotation device adapted for rotating the device so that the sensors pass over the one or more optical sensing devices, the method comprising: providing a fluid at an inlet near the centre of the device, rotating the device, and obtaining properties of the sensors using the optical sensing devices.

### FIELD OF THE INVENTION

The present invention relates to an apparatus for analysing samples and methods for analysing samples.

### BACKGROUND OF THE INVENTION

Test devices, such as described in WO 2006/122360, are used for performing analysis and test of chemical compounds, e.g. identification of compounds in liquids.

Present test systems suffer from low throughput and hence, an improved device and measurement method would be advantageous, and in particular a more efficient and/or reliable generation of multi-parameter data would be advantageous.

It is one object of the present invention to provide an alternative to the prior art.

It may be seen as an object of the present invention to provide a test system for performing multiple parallel analyses, providing a higher throughput and thus a more efficient system. Such a system solves the above mentioned problems of the prior art and provides a much improved testing system and method.

### SUMMARY OF THE INVENTION

Thus, the above described object and several other objects are intended to be obtained in a first aspect of the invention by providing an apparatus for detecting compounds. The apparatus having a device defining a disk-shaped geometry, the device having a centre, a plurality of fluid channels each comprising a fluid inlet positioned at a first distance from the centre and a fluid channel end at a second distance from the centre, the second distance being larger than the first distance, one or more sensors arranged at each fluid channel, wherein the sensors each comprise at least one optical detectable member, the test apparatus further comprising one or more optical sensing devices arranged for sensing the at least one optical detectable member of the one or more sensors, and a rotation device adapted for rotating the device so that the sensors pass over the one or more optical sensing devices. The optical sensing devices is configured or adapted to sense or detect properties of the sensor, i.e. the optically detectable member. The sensors may individually be arranged so as to pass over one or more of the optical sensing devices. Further one or more of the optical sensing devices may be adapted to be moveable so as to perform measurements on one or more sensors at different distance from the centre of the device.

The fluid channels may advantageously be substantially straight lines from the centre of the device. One or more of the fluid channels may include an extended area or volume i.e. a test chamber as described below. The inlet of the fluid channel is preferably all positioned at the same distance from the centre of the disk-shaped device. The fluid channel end may be a reservoir for collecting residual fluid. Alternatively an outlet port may be provided so that fluid may be extracted or discarded. The fluid channels may include capillary valves.

Advantageously the optical, or optically, detectable member in the sensor may include a beam. Advantageously the optical detectable member in the sensor may include a cantilever beam. Alternatively the optical detectable member in the sensor may not include a beam but be a Surface-enhanced Raman Scattering (SERS) substrate.

The test apparatus is configured or adapted to rotating the device after a sample is introduced. The device is preferably disk-shaped, i.e. circular and substantially flat. The device is a carrier having one or more fluid channels. When the device is rotated the sample is moved from the initial position near the centre of the device towards the rim of the device due to centrifugal forces and/or capillary forces.

The rotation device may be an electro motor having a belt drive coupled to the device, i.e. the disk-shaped carrier.

Advantageously two or more sensors may be arranged in a fluid channel at different radii. By positioning two or more sensors, e.g. three, four, five, six, seven, eight, nine, ten, or even more sensors at different positions in a fluid channel several measurements are possible in the same operation, thereby allowing higher throughput of measurements. The measurements may include determination of the presence of a specific compound. Corresponding optical sensing devices may be positioned at the radii where the sensors are located in the fluid channels. Alternatively one or more optical sensing devices may be placed on a movable mount so as to move the optical sensor between one or more positions where measurements are to be performed.

Advantageously each of the sensors are arranged in a test chamber in fluid communication with a respective fluid channel. The test chamber may be an enlarged area in the channel, e.g. a space or cavity, where a sensor is positioned

Advantageously the optical sensing device is arranged so as to sense deflection property, surface property and/or frequency property of the at least one beam. The sensors may include a multitude of beams as described in more detail elsewhere in the present description. Advantageously between 15 and 30 beams are used per sensing chamber, such as 24 beams. Advantageously between 6 and 30 chambers or cavities are used in one device, which corresponds to between 144 and 720 beam in a device.

Advantageously at least one of the optical sensing devices may be arranged for detecting wobbling of the device and a controller for determining corrective values for the optical sensing device. By detecting wobbling of the device, i.e. when the device is spun, is useful as irregularity of the device and/or misalignment of the device relative to the optical sensing device may lead to misinterpretation of the measurements.

Advantageously the sensor, e.g. the one or more beams, includes receptors, DNA strands, antibodies, antigens or enzymes which will selectively attract and bond with the particular substance to be detected. As mentioned when a compound to be detected is bound to a beam, the properties of the beam will change and the amount and/or presence of the compound may be determined.

Advantageously at least one of the optical sensing devices have an optical input having a numerical aperture in the interval 0.1 to 0.85. When using a commercially available optical sensing device, e.g. an optical pick-up head of a DVD player or the like, it is advantageous to adapt or modify the numerical aperture of the lens in the optical device so as to obtain an optimal detection of the sensor, i.e. the beams.

Advantageously the apparatus may include a connection to a computer device allowing the position of the sensors to be determined and displayed by the computer. Further the computer device may be used for controlling the apparatus and its components, e.g. the computer device may provide a graphical user interface. The computer device may be used for collecting data from the optical sensing devices. A storage device may be provided in the apparatus so as to collect and store data from the different sensors.

Advantageously at least one of the optical sensing devices may include a first and a second optical receiver, wherein the first optical receiver is adapted for determining and compensating wobbling of the device and the second optical receiver is adapted for determining properties of the sensors. By having two optical receivers, the two may be used for different purposes.

Advantageously the device includes a patterned ring and the first optical receiver is adapted for calibration by detecting the patterned ring. This patterned ring may be used for calibration purposes. The patterned ring may be a circular track in the device.

Advantageously the sensors are read, or measured, using astigmatism and the apparatus comprises an optical read head from a CD-player, a DVD-player and/or a Blu-ray player. It is contemplated to be advantageous to use an existing system having an optical reader i.e. an optical pick-up head.

A second aspect of the present invention relates to a method for determining compounds comprising the steps of providing an apparatus for detecting compounds having a device defining a disk-shaped geometry, the device having a centre, a plurality of fluid channels each comprising a fluid inlet positioned at a first distance from the centre and a fluid channel end at a second distance from the centre, the second distance being larger than the first distance, one or more sensors arranged at each fluid channel, wherein the sensors each comprise at least one optical detectable member, the test apparatus further comprising one or more optical sensing devices arranged for sensing the at least one optical detectable feature of the one or more sensors, and a rotation device adapted for rotating the device so that the sensors pass over the one or more optical sensing devices, the method comprising providing a fluid at an inlet near the centre of the device, rotating the device, and obtaining properties of the sensors using the optical sensing device.

The method may provide high throughput analysis of samples with multiple, parallel measurements.

Advantageously the apparatus used for the method may include any of the features of the first aspect.

Advantageously the method may further comprise determining one or more of: deflection, resonant frequency, surface roughness, and/or thermal noise of sensors. A combination of detection of more than one property may increase the reliability and/or precision of the detection/measurement.

Advantageously the optical sensing device includes a first and a second optical receiver, wherein the first optical receiver is adapted for determining wobbling of the device and the second optical receiver is adapted for determining properties of the sensors, and the method may comprise calibrating the optical sensing device using the signal from the first optical receiver

The invention is particularly, but not exclusively, advantageous for obtaining a test system for testing fluids and determining compounds in the fluids.

The apparatus according to the present invention have small dimension and/or weight compared to related products.

It is contemplated that the apparatus and method according to the present invention will provide extremely cost reduction of the final product, while maintaining high throughput beam reading. With the present invention around 20000 beam measurements can be done in 1 minute compare to 1 single measurement in 15 minutes with traditional systems in comparative conditions (i.e. using the system according to the present invention at 1 hertz spinning).

The apparatus according to the present invention will provide greater, i.e. improved, precision of the measurements.

The apparatus according to the present invention will allow measurements to be performed with flexibility: possibility of coating beams of the same device/disk with different chemistry: will allow sensing several biochemical compounds with a single, compact, low cost platform.

When using the apparatus according to the present invention it is contemplated to allow easy replacement of the sensing tools: it will be as easy as changing a DVD from the player.

Advantageously the beams are cantilever beams or beams supported at more than one side or end, e.g. doubly clamped beams.

The apparatus according to the present invention provide a unique system allowing a number of different measurements in one single platform, e.g. 3 different measurements: bending, thermal noise and roughness. This is not possible in existing commercial products.

In the apparatus according to the present invention the Raman peak intensity may be significantly enhanced when the SERS substrate is integrated in this specific design giving more sensitive results which is important for detecting trace of chemical compounds in the air for example.

The first and second aspects of the present invention may each be combined with any of the other aspects and features mentioned in relation to any of these aspects may be combined in any possible ways. These and other aspects of the invention will be apparent from and elucidated with reference to the embodiments described hereinafter.

### BRIEF DESCRIPTION OF THE FIGURES

Embodiment of the apparatus and method according to the invention will now be described in more detail with regard to the accompanying figures. The FIGS. show one way of implementing the present invention and is not to be construed as being limiting to other possible embodiments falling within the scope of the attached claim set.

FIG. 1 is a schematic illustration of parts of a system according to the present invention,

FIG. 2 is a schematic illustration of a system and measurements,

FIG. 3 is a schematic illustration of measurement results for protein detection,

FIG. 4 is a schematic illustration of measurement results for antibodies detection,

FIG. 5 is a schematic illustration of a device according to the present invention,

FIG. 6A and 6B are schematic illustrations of a block diagram of systems according to the present invention,

FIG. 7 is a schematic illustration of the principle of astigmatism,

FIG. 8 is a schematic illustration of a sensor having cantilevers,

FIG. 9 is a schematic illustration of optical sensors having different numerical apertures,

FIG. 10 is a schematic illustration of different measurements,

FIG. 11 is a schematic illustration of a device according to the present invention,

FIG. 12 is a schematic illustration of details of a measurement setup,

FIG. 13 is a photograph of a disk for a test system according to the present invention,

FIG. 14 is a photograph of a part of a device and an optical pick-up head,

FIG. 15 is an image of an optical sensing device having two optical receivers,

FIG. 16 is a schematic illustration of a cantilever sensor,

FIG. 17 is an image of a part of a sensor having multiple beams,

FIG. 18 is a schematic illustration of a fluid channel,

FIG. 19 is a schematic illustration of a device having a number of fluid channels,

FIG. 20 is an image of a part of a fluid channel,

FIG. 21 is a schematic illustration of a device and close-up illustrations of parts of the device, and

FIG. 22 is a schematic illustration of the calibration using an optical sensing device having two optical receivers.

### DETAILED DESCRIPTION

Cantilever-based sensors have for more than 15 years been studied as a tool for label-free sensing. Molecules bind to cantilevers and cause the cantilevers to bend and/or the resonant frequency to change. These sensors have been limited in terms of few data sets and little statistics. We propose to use optics and mechanics from a regular DVD player to handle liquid samples and to read-out cantilever deflection, resonant frequency and surface roughness. More than 1000 cantilevers can be read per second and the approach was used to detect the specific binding of streptavidin and antibodies. We see the DVD platform as an instrument to achieve high volume data sets facilitating the use of cantilever-based sensing in high throughput label-free sensing.

Micrometer and even nanometer sized cantilevers have since the mid-1990s been studied and used for label free molecular recognition. For molecular recognition the cantilever is typically functionalized with probe molecules designed to specifically bind certain target molecules in solution. The specific binding of target molecules causes the cantilever to deflect due to a change in surface stress. Alternatively, the mass change of the cantilever can be monitored by measuring the resonant frequency change of the cantilever because the resonant frequency is inversely proportional to the added mass.

Today, the prevalent method of monitoring vibrational amplitudes and cantilever deflection is based on the optical leverage technique widely used in atomic force microscopy8. Such systems are typically bulky because of the requirement for a long optical path. Also, the focusing of the laser spot on the cantilever and the alignment of the laser beam on the optical detector are tedious and time consuming. Alternatively, a CCD camera has been used for monitoring cantilever deflection and hereby large 2-dimensional arrays of cantilevers can be read simultaneously with a deflection resolution of approximately 1 nm 9. However, the method requires that all cantilevers are in the same focal plane which is extremely difficult to achieve in practice. Both techniques only apply to micrometer sized cantilevers since the spot size in the optical leverage systems is typically 20 □m or above and since the intensity of the reflected light is otherwise too low in the CCD system. Integrated read-out has been suggested by several groups. For example cantilevers with piezoresistive, piezoelectric and MOSFET-based read-out have been developed and applied for molecular recognition. Generally, these cantilevers have to be carefully insulated in order to be operated in liquid and the devices require significantly more packaging due to electrical interconnections. The reported signal-to-noise ratios are in most cases at least a factor of 10 lower than for optical leverage.

Typically, the cantilevers are placed in small polymer or ceramic chambers and different liquids are introduced using i.e. syringe pumps. The pumps are a potential noise source and the liquid handling is tedious and slow. Finally, few papers on cantilever-based sensing present statically analyzed data sets—probably because cantilever sensing is normally performed on one or maybe two cantilevers at a time (one for reference) and a single measurement is rather elaborate and time consuming, primarily because of the instrumentation.

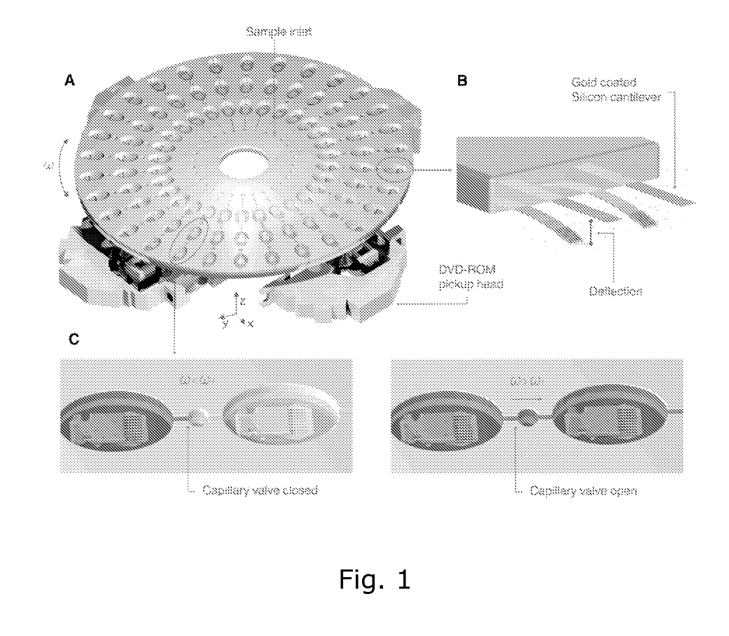

We report on a DVD based sensor platform that reduces the aforementioned obstacles and challenges in cantilever based sensing. The concept is illustrated in FIG. 1. A DVD shaped disk is used to mount up to 90 cantilever chips, each with 8 cantilevers, in a radial symmetry. In this work silicon cantilevers with a length of 500 μm a width of 100 μm and a thickness of 1 μm have been used16. All cantilevers are coated on the top side with a nm thick gold layer. The disc is 25 structured in Pyrex and the polymer SU-8 and contains holding substrates for the cantilever chips. The cantilever chips are simply clicked into the holding substrates after functionlization and can be replaced by tweezers without significantly damaging the chips. This leads to a flexible sensing system, where differently functionalized chips can be interchanged depending on the analytes to be detected. Approximately 1 mm below the disk four DVD-ROM optical pickup heads (PUHs) provide the read-out system. The disk is spun and cantilevers are illuminated by the DVD lasers with a wavelength of 650 nm and a spot diameter of only 0.56 μm (FWHM). The deflection profiles are measured using the astigmatism-based detection mechanism normally used for auto focusing. The PUH can measure the cantilever profile with a resolution better than 1 nm in Z direction allowing precise and automated 3D reconstructions of the cantilever surfaces. We have measured cantilever deflections at rotating velocities up to 120 rpm, which equals to more than 1000 cantilevers per second. At present, typical measurements are performed at 0.1-2 rpm (1-20 cantilevers per second). The DVD disc format has in the past 10 years been widely used for liquid handling. By spinning the disc the generated centrifugal forces can be used to move liquid from the inner part of the disc and towards the outer rim. In our design liquid can be handled using capillary valves which burst at certain frequencies. These allow precise sample dispensing to the reservoirs where the cantilever chips are clamped.

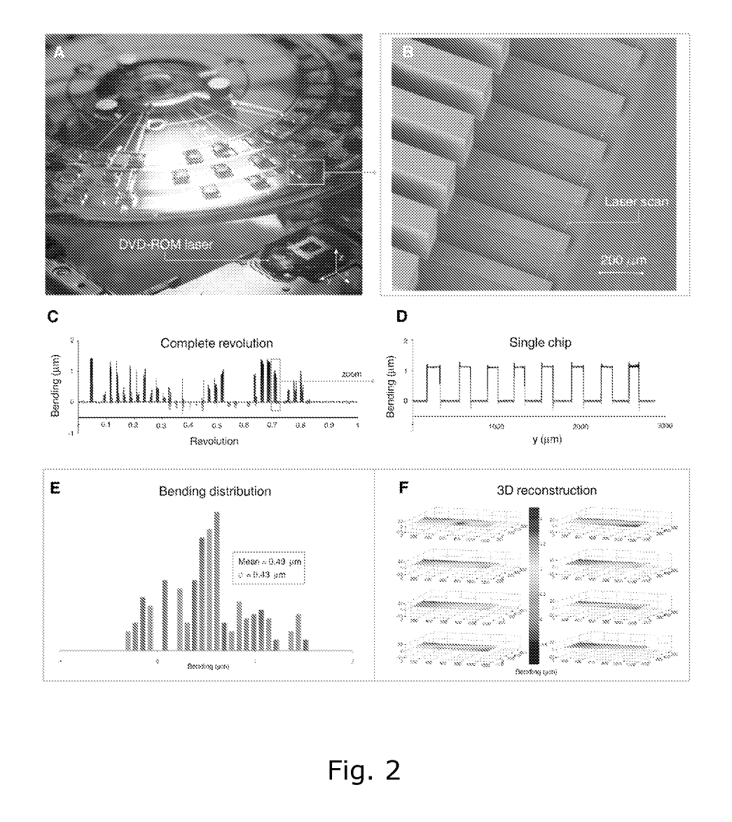

A photograph of the realized DVD platform with mounted cantilever chips is shown in FIG. 2A. A reflective aluminum pattern on the disk surface ensures that the DVD-ROM PUH maintains the focus distance. The laser scans from the bottom, passing through the glass substrate and focuses on the cantilever surface (FIG. 2B). Typical sampling rate corresponds to around 1000 measurement points across the width of each cantilever. We thus obtain a profile where data points are acquired every 100 nm along the width of the cantilever.

An example of raw signal acquired during one revolution of the disk is shown in FIG. 2C. The plot is composed of around 1.000.000 data points. Each peak represents a chip (composed of 8 cantilevers). Typical experiments include or consist of 30-50 revolutions, resulting in up to 50 million measurement points. Strong data processing is thus required in order to extract the useful information from the large amount of data. Zooming in on FIG. 2C we can extract the individual cantilever profiles, as seen in FIG. 2D. Knowing the rotating velocity it is possible to convert the Y axis to a traveling distance.

Before sensing experiments are performed, each cantilever is fully characterized by at least 10 measurements (10 revolutions of the disk). The variance of the measurements is used to evaluate the reliability of the measurements. Typically, the standard deviation after 10 measurements is below 10 nm. The noise is typically higher at the outer 10-15 microns of the cantilever profile, and this region is therefore generally removed before data processing. Once the data process is performed it is possible to obtain a detailed statistical analysis of the initial conditions of the cantilevers in air. The histogram in FIG. 2E shows the distribution of initial cantilever bending from 30 chips (240 cantilevers) measured over 10 revolutions. The average bending is 0.49 μm, with a standard deviation of 0.43 μm

An example of eight reconstructed cantilever surfaces from a single chip is shown in FIG. 2F. The 3D reconstruction gives valuable information on the roughness of the cantilever surface. In our work, the roughness is used to evaluate the distribution of biomolecules on the cantilever surface. When inhomogeneous binding of material occurs, the optical properties (refractivity, reflectivity) change, giving rise to a “rough” optical profile. When monolayer-type binding occurs, the optical profile of the surface appears smooth.

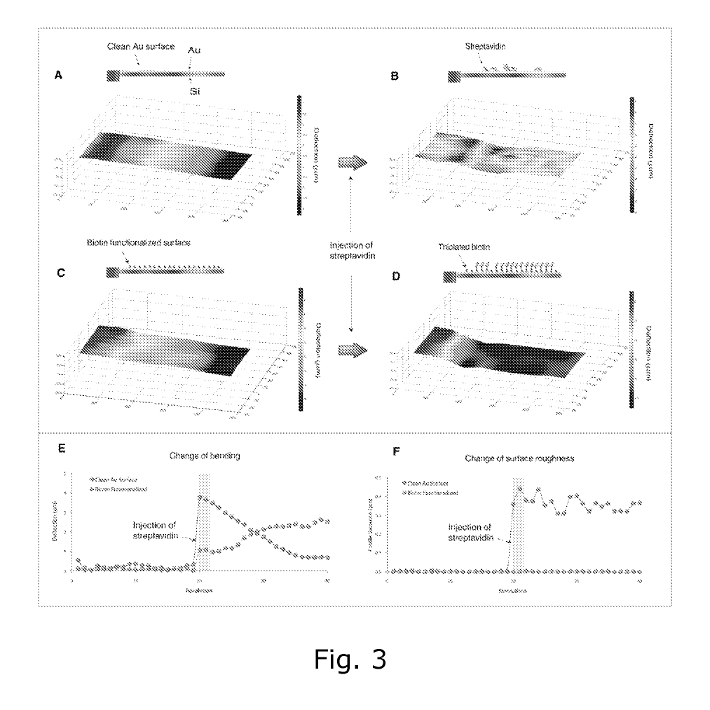

For biomolecular binding experiments, 8 cantilevers were functionalized with thiolated biotin and 8 untreated cantilevers were used for reference measurements. Next, the chips were inserted into the DVD platform and exposed to a buffer solution containing streptavidin (concentration??). After exposure, all cantilevers were gently washed in deionized (DI) water in order to remove any residual salt from the buffer solution. After washing, the water was left to evaporate and the cantilever responses were measured continuously. FIG. 3A shows the averaged 3D reconstruction of 8 untreated cantilevers, measured before the injection of streptavidin into the cantilever reservoir. The surfaces have a low roughness of a few nm, indicating that the gold layer is clean. The initial deflection (at the cantilever apex) is around 5 μm. After the injection of streptavidin and a washing step the same cantilevers show a high increase in the surface roughness, indicating that an inhomogeneous layer has been formed. Additionally, the deflection of the cantilever has changed approximately 1 μm. Both observations can be explained by unspecific binding of streptavidin to the cantilever surface.

The cantilevers functionalized with biotin are initially bent 6-7 μm at the cantilever apex and the surface appears optically smooth, see FIG. 3C. This suggests that the biotin functionalization has created a monolayer on the gold surface of the cantilevers. After the biotin-streptavidin binding has occurred, the observed change in cantilever bending is approximately 3 pm and the roughness of the surface appears unchanged, indicating that streptavidin has been uniformly bound to the biotin layer.

In FIG. 3E a statistical analysis of the change in the bending of the cantilevers is shown. Each data point corresponds to the averaged value from 8 cantilevers. We notice, that after the injection of streptavidin the bending of the untreated cantilevers decrease, reaching an asymptotic value after around 15 disc revolutions (corresponding to approximately 5 minutes). At this stage the water has fully evaporated and stable measurement conditions can be obtained. Similar behavior (but opposite direction) is observed for the biotin functionalized cantilevers. The biotin functionalized cantilevers have an averaged deflection which is approximately 2 μm larger than for the untreated reference cantilevers when the measurements have stabilized. The averaged change in surface roughness (FIG. 3F) is significant for the untreated cantilevers compared with the functionalized ones. This change is faster than the bending, indicating that the evaporation of the water does not affect the distribution of biomolecules on the gold surface. A roughness change is also observed for the biotin-functionalized cantilevers—however it is almost 2 orders of magnitude lower than for the reference cantilevers.

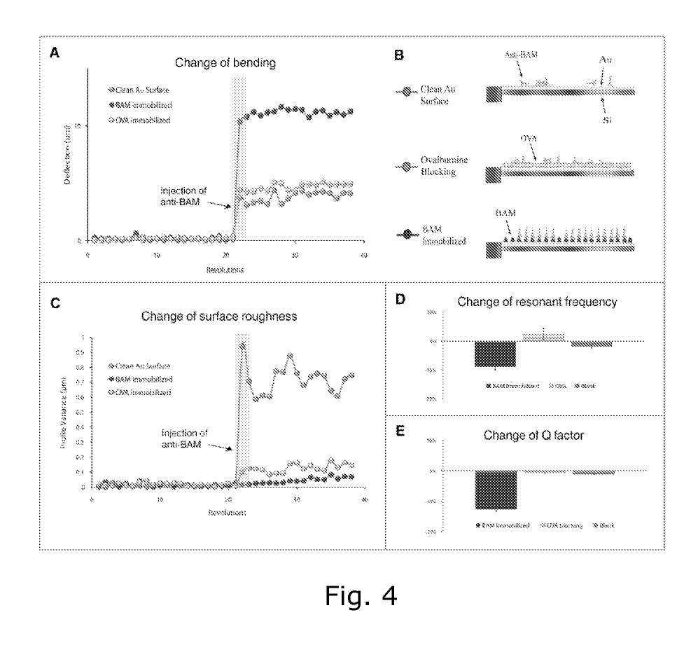

Similar experiments have been performed for detection of the pesticide derivative 2,6-dichlorobenzamide (BAM). The used protocol has been developed for a competitive assay which implies that the sensing cantilevers are initially coated with a layer of BAM 23. As antibodies against BAM bind to the surface the cantilever is anticipated to bend. Two chips have been prepared for the measurements, each containing 2 cantilevers functionalized with BAM, 2 cantilevers with an ovalbumine blocking layer and 4 untreated cantilevers. The initial bending of the cantilevers is measured as above and specific antibodies against BAM are injected into the cantilever reservoirs followed by a rinse in DI water and subsequent water evaporation. FIG. 4A shows the induced averaged bending of the differently functionalized cantilevers. The BAM-functionalized cantilevers deflect approximately 10 μm compared with 3-5 μm for the blank and ovalbumine coated cantilevers. Probably, the antibodies bind strongly to the BAM functionalized surfaces causing a large change in surface stress whereas they bind unspecifically to the other cantilevers, illustrated in FIG. 4B. Cantilever profiles reveal that the untreated cantilevers become significantly rough, while the BAM and ovalbumine coated cantilevers are unaffected by the introduction of antibodies. The ovalbumin coated cantilevers are initially rough reflecting the nature of the coating, see FIG. 4C. We believe that this is once again an indication that specific binding results in ordered uniform layers whereas the unspecific binding results in a random and rough surface.

In the BAM experiments we have also tested the capability of the system to measure changes in the resonant frequency using the thermal noise peaks of the cantilevers24. FIG. 4D shows the change in percentage of the resonant frequency of the 16 cantilevers after the reaction with antibodies has taken place. The BAM functionalized cantilevers have the highest negative change in resonant frequency (approximately 10%), indicating that mass has been added to the cantilever. The ovalbumin blocked and untreated cantilevers have minor changes in the resonant frequencies (1-2%). This smaller change can be attributed to unspecific binding of antibodies as wells as solidification of salt present in the buffer solution. The ovalbumin coated cantilevers have a positive change which might be a result of changes in both added mass and surface stress. The corresponding Q-factors of the cantilevers can be extracted from the resonant curves (FIG. 4E) and they generally follow the changes in resonant frequency.

The DVD platform offers a number of advantages over traditional cantilever sensing. It readily supplies large amount of data for statistical analysis facilitating the onset of statistical cantilever based sensing. Moreover, the platform allows for simultaneous measurements of deflection, vibrational amplitude and surface roughness improving the amount of information to be achieved and consequently the reliability of data.

FIG. 1. (A) Schematic of the DVD-ROM platform for cantilever-based sensing. High throughput sensing as well as liquid handling are achieved by spinning the disk. (B) Chips, each containing eight gold-coated cantilevers, are mounted on the DVD shaped substrate. (C) The chips are clipped onto the substrate and the liquid flow is controlled by capillary valves which burst at a certain threshold frequency.

FIG. 2. (A) Photograph of DVD-ROM platform with integrated cantilever chips. The disc is fabricated in glass and the polymer SU-8. (B) Scanning Electron Microscope image of gold-coated silicon cantilevers with dimensions 100 μm×500 μm×1 μm. (C) Raw data from one revolution of the DVD. Each peak corresponds to one cantilever chip. (D) The obtained profiles from a single cantilever chip. (E) Distribution of the measured initial bending of the silicon cantilevers. (F) Example of 3D reconstruction of eight cantilever surfaces from the same chip.

FIG. 3. (A) Surface reconstruction of gold-coated silicon cantilever. (B) The same cantilever after exposure to streptavidin solution. The roughness is seen to increase. (C) Surface reconstruction of biotin functionalized cantilever and (D) of the same cantilever after reaction with streptavidin. The roughness and deflection are changed significantly. (E,F) Averaged change in cantilever bending and surface roughness for an untreated gold surface (average value of 8 cantilevers) and a biotin functionalized surface (average value of 4 cantilevers). The blue region indicates the time the cantilever is in contact with the streptavidin solution. After approximately 15 revolutions the bending signal stabilizes and the resulting difference in deflection is approximately 2 μm. The surface roughness is unchanged for the biotin functionalized cantilevers whereas it drastically and rapidly increases for the untreated gold surface.

FIG. 4. (A) Averaged changes in cantilever deflections when exposed to BAM antibodies. All data points represent averaged values from either 4 (ovalbumin and BAM coated) or 8 (untreated gold-coated) cantilevers. (B) Graphical representation of the differently coated cantilevers. (C) Averaged changes in surface roughness after exposure to BAM antibodies. The ovalbumin and BAM coated surfaces are basically unchanged whereas large and rapid changes are seen for the gold coated cantilevers. (D,E) Measured averaged changes in resonant frequency and Q-factor . They are seen to drop significantly for the BAM coated cantilevers indicating binding of the BAM antibodies.

The invention includes the integration of four different sensing technologies into a compact, highly sensitive and high throughput single platform. This invention is designed to achieve levels of sensitivity impossible to obtain employing a single-technology based sensor. Biochemical analysis, water control, environmental monitoring, detection of hazardous compounds, both in air and liquid, are suitable applications for our technology.

Our system is based on the integration between DVD-ROM utilities technology, micro-cantilever based sensors, SERS spectroscopy, colorimetric chemical arrays, and spin-based capillary valves technology.

The serial organization of the four sensors, in other embodiments other numbers of sensors are possible, allows the multiple analysis of the same sample, consisting in few microliters of fluid in form of pre-concentrated buffer solution (for measurements in air) or of bio-chemical sample (in case of liquid measurements), leading to a highly increased sensing accuracy. The sample sensing order can be easily inverted or modified in each platform, depending on the biochemical reactions induced in the different sensing reservoirs. Ten or more parallel sensing lines are integrated in the same platform, thus several measurements can be performed simultaneously on the different sensors, leading to a highly flexible and powerful detection system. The complete platform has dimensions comparable with a compact disk (CD).

The readout systems are designed to be compact and robust, in order to allow the device to be easily handled and to reduce the risk of miscalibration during transport processes. Numerical adjustments and calibrations of the mechanical and optical components are employed to compensate the errors induced by external events.

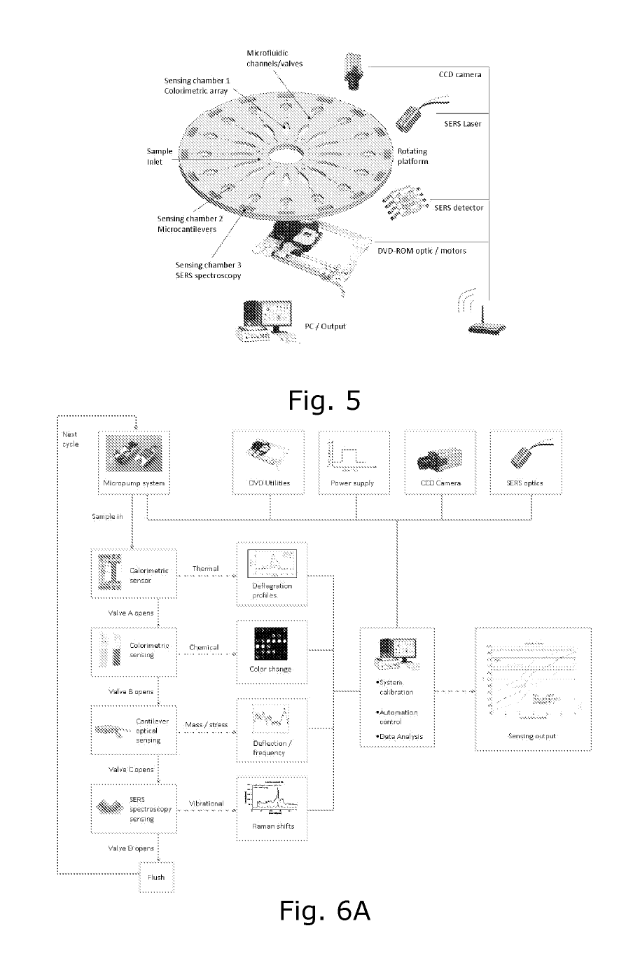

FIG. 5 illustrate the general layout of a system, having or consisting of two main blocks: (i) the rotating platform, composed by a microfluidic substrate, the holding substrate, the colorimetric array chips, the SERS nanograss chips and the 25 microcantilever chips; (ii) the readout system, composed by a CCD camera, the DVD-ROM utilities, and the SERS optical system. The signals obtained by the optoelectronic readout components are sent wireless to a computer in order to be digitally analyzed and treated. It is possible to add a calorimetric bridge sensor and a corresponding electronic block, this is not illustrated in the figure.

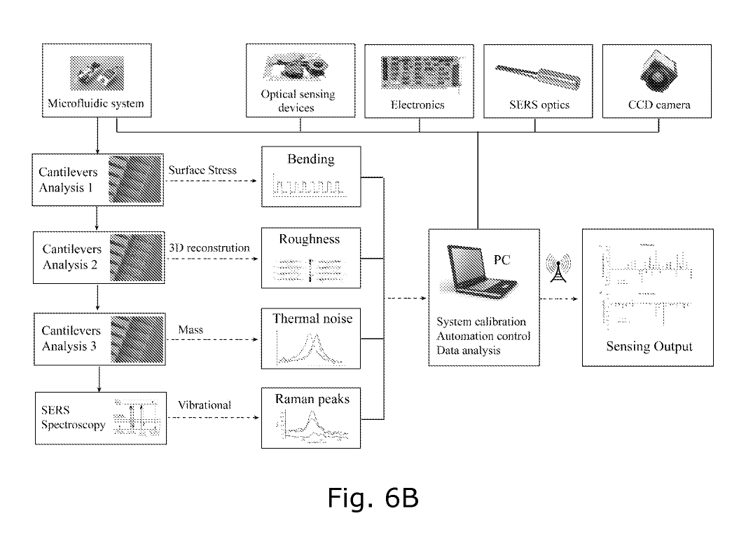

The working principle of the complete device is depicted in FIG. 6. First of all, a microfluidic system (composed by pumps, needles and an electronic stage) drives 10 μL of sample into each channel. In each line the sample is driven by centrifugal force through the microchannels into the sensing reservoirs, separated by microcapillary valves that can be opened spinning the platform at certain angular frequencies. Once the desired reaction has been taken place in the first reservoir, the sample will move into the next sensing chamber increasing the spinning velocity of the motor and the second reaction will take place. After the last sensing chamber has been filled and the last sensor covered by the sample liquid, the platform is spun at high frequency (1200 rpm) and the fluid is washed out from the channels and the reservoirs, leaving the system clean and ready to start a new analysis. It is estimated that each cycle time will be of the order of few minutes.

At the end of the entire cycle, each line will provide 4 different analysis (thermal, chemical, vibrational and stress induced) of the same microvolume of sample. It is also important to remark that if we consider that each platform, in one embodiment, will consist in 30 lines, 120 different sensing measurements will be performed at each revolution of the platform. So, if the disk is spun at 1 Hz will lead to 7200 analysis per minute.

The combination between capillary forces and centrifugal force makes possible to design the microfluidic channel in order to provide a pressure barrier capillary-induced equal to the one induced (in opposite direction) by spinning the platform at a given angular frequency, making possible to move the liquid into serial chambers tuning the angular frequency of the platform.

In the first sensing chamber, where the thermal response of the analyte to the temperature change due to melting, evaporation, decomposition or deflagration of the sample is monitored. The signal gives a unique signature for different analyzed compounds.

The sensor includes or consists in a micro heater designed as a bridge, fabricated using standard cleanroom processing techniques. The bridge is made of silicon nitride with integrated heating elements and temperature measurement resistor made of doped silicon. Two microheaters are combined in a differential thermal analysis (DTA) system making calorimetric measurement possible. The electric contacts of the sensors will be connected by removable pins, after the platform has been stopped. A single thermal measurement takes around 100 microseconds to be performed.

The second sensing chamber provides a chemical analysis of the sample based on the ability of certain molecules to change the color when reacting with specific analytes. The monitoring of the color change is obtained through frame capturing the microarray of sensors (96 spots) at each revolution, and treating numerically the data acquired. A CCD camera with integrated image analysis software is employed in the system.

In the third sensing chamber is monitored the stress induced by the binding of specific molecules to a selective surface of a microcantilever beam. Furthermore the change in the resonant frequency due to mass absorption on the cantilever can be measured.

One of the important components for the initial implementation of the present invention was the DVD-ROM setup for the readout analysis and motor control.

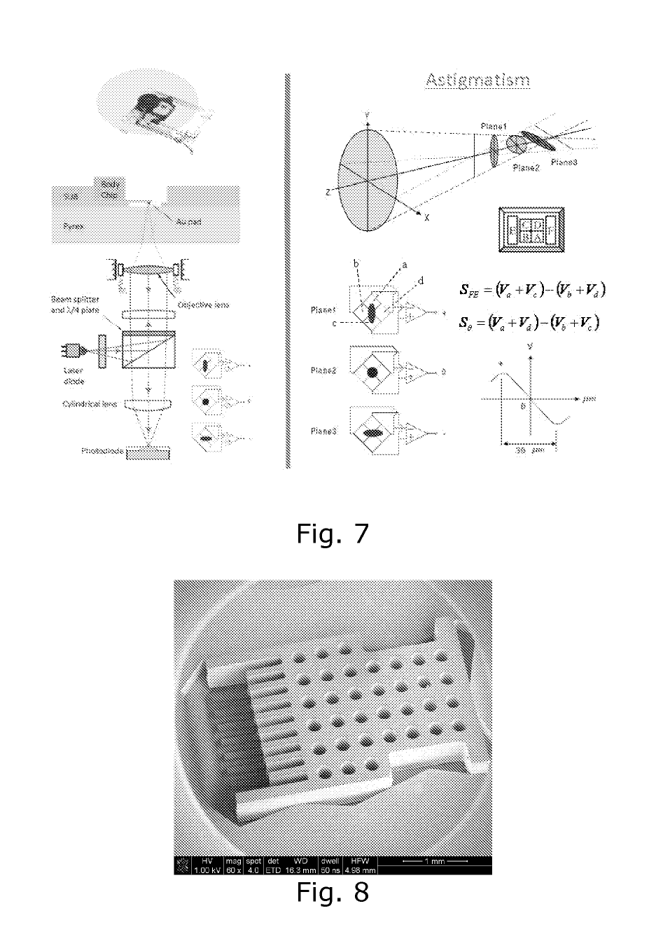

FIG. 7 illustrates the cantilever readout principle based on DVD-ROM technology. The deflection of the cantilever beam is measured through the Focus Error signal (FE) obtained differentiating the laser intensities on the four quadrants composing the photodetector. The asymmetry of the laser beam shape is obtained by inducing astigmatic aberration in the optical system. This aberration is induced by cylindrical lenses integrated in the DVD-ROM pickup head optics. Detection of Sub-nanometric displacements of the cantilever are achievable with this type of optical method.

Once the rotating motor is spun, sequential profile analysis of the cantilevers can be performed, together with resonant frequency measurements. The measured profile signal can be averaged over data acquired at each revolution of the platform. Statistical and numerical signal processes of the signals lead to and increased signal to noise ratio and in general to a higher sensitivity to the deflection of the beam.

The substrate-chips system has to be accurately aligned and centered with respect of the rotational axis, and the cantilevers have to be well clamped and parallel oriented to the surface of the disk, as shown in the SEM picture in FIG. 4.

The last sensing process is based on SERS technology. The SERS substrates developed at Nanotech have shown top class properties and application opportunities.

The integration of the Raman analysis into a rotating platform has shown great opportunities in enhancing the Raman peaks intensity. In fact the dynamical readout leads to a statistically larger chance of laser hitting the analyte molecule on the substrate. This is an important issue when trace levels of chemical compounds are has to be monitored. Furthermore it is observed sharper spectra of the vibrational frequencies. Rotating the platform avoid the overheating of the hotspots, hence preventing peak broadening to occur.

After all the signals are obtained, a numerical analysis of the data is needed. The integration of the different sensors provides a very high increase in the sensitivity of the system to one (or more) specific target.

Under complete independence, a clearance efficiency of 60-90%, and a relatively low false alarm rate, the clearance efficiency of the combined system will increase exponentially with the number methods applied to the same area. The false alarm rate, however, will only increase linearly.

Achieving more than 99% efficiency can thus be obtained by applying a few methods, while keeping the false alarm rate low. Even with some dependence among methods it is possible to device a combination strategy which always ensures that the efficiency of the combined system is higher.

Another advantage of combining methods is increased robustness to changing environmental conditions and assumptions.

One of the technologies implemented in the system is the modification of the optical path of the DVD-ROM/Blue-Ray pickup heads.

In order to be able to scan hundreds or thousands of cantilever sensors mounted on the rotational platform, the linear working range of the Focus Error Signal (FES) needs to be tuned.

In fact, using the commercial devices without modification it is impossible to perform high-throughput analysis. This is due to the intrinsic incompatibility between the initial bending of the cantilever sensors (from ±1 μm to ±10 μm), the mechanical wobbling of rotating stages (from ±20 μm to ±500 μm), and the short linear range of commercially designed optical heads (from 2 μm to 6 μm). With commercially available devices it is not possible to monitor the deflection, the roughness and the thermal noise in liquid medium.

Furthermore it is not possible to employ the auto-calibration mechanism that is included in the commercial devices. In fact, if the FES is used for measuring the cantilevers, it cannot be used for auto-tracking the wobbling of the disc. The auto-tracking system measures the variation of the distance between the focal point and the pickup head, thus possible information about the bending of the cantilevers would be suppressed by the re-adjustment of the built-in auto-focusing mechanism.

The apparatus includes a mechanical modification (substitution) of the objective lens of the pickup head of a commercially available unit. We optimized the modification process in order to find the optimal Numerical Aperture (NA) of the lens for specific sensing processes. We are able to tune the optical working range of the FES from few pm up to 350 μm, using lenses whose NA varies from 0.1 to 0.85. We can control the focus distance, the sensitivity of the detection, and the performances of the optical path to work in liquid or in air.

In this way we are able to monitor the deflection, the surface roughness, and the thermal noise of cantilevers loaded on the rotational platform independently on their position of the disc. We can spin the disc very fast, and every cantilever would then lie within the working linear range of the modified optical path. This is also a key technology feature to be able to measure in liquid.

With this technology we can achieve sensitivities of the order of few nm/mV when measuring hundreds of cantilevers per second in liquid medium. Depending on the conditions, sub-nm resolution can be achieved implementing this methodology.

In one approach we develop our technology by using a Blue-Ray optical pickup head to make ultra-high resolution measurements combined with ultra-fast cantilever scanning.

We employ a Blu-Ray disc pickup head which has 2 objective lenses mounted on its moving structure. One lens is originally designed to read DVDs, the other to read Blu-Ray discs.

In our technology we employ both lenses for calibration purposes. The Blu-Ray device (NA=0.85) is focused on a specifically designed patterned ring (coated with reflective material, e.g. Al or Au) and its built-in auto-tracking system is employed to keep the double-lens structure at constant distance from the disc. In this way the wobbling of the rotating stage, even if greater than the working FES range, could be compensated. The second lens (the DVD-ROM one) is then used for scanning the cantilevers and to measure the deflection, surface roughness and thermal noise through the values obtained via the DVD-ROM Focus Error Signal. The Blue-ray pickup head has resolution of hundreds of picometers, thus allowing extremely accurate auto-tracking of the system wobbling. The DVD-ROM lenses, modified according to the previous part, could then be tuned to give extremely accurate and fast analysis of the cantilevers. In this way we can measure simultaneously thousand of independent cantilever sensors with sub-nanometric resolution and with very high speed (up to 1000 cantilever per second).

An approach for wobbling compensation was developed modifying the rotating stage and including a mechanical bearing with high-precision rotational properties. Using this approach we can implement the calibration methods and the optical modification explained in the previous sections into the same, high-throughput and high-resolution readout device.

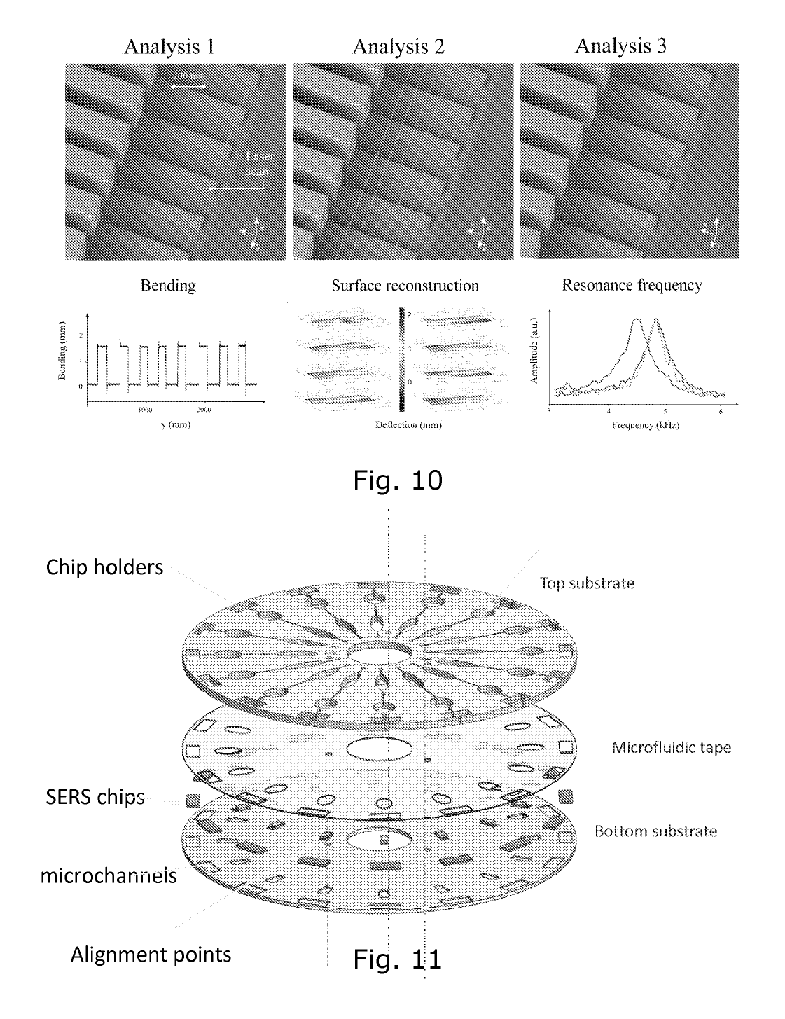

The astigmatic detection method is a powerful and versatile tool for monitoring the deflection of cantilever beams, as well as to measure their surface properties and their resonance frequencies. The working principle of the DVD-ROM based readout applied to cantilever sensors is schematically illustrated in FIG. 10.

In the device, the cantilever chips are mounted on the rotating disc keeping the sensors suspended over a glass window. The laser beam is positioned at a distance from the cantilever apexes that fall inside the linear range of the Pick-Up Head PUH (a configuration that may be obtained through manipulation of the optical path). When the device, i.e. the disk, is spinning, the laser scans the cantilever beams acquiring the Focus Error Signal generated by the laser spot shape on the PDIC. When the laser path crosses the gap between cantilevers, no signal is acquired due to the lack of reflective material. On the other hand, when the light shines onto the cantilevers the FES is measured and the cantilever signal is acquired.

The signal is thus an array of profiles spaced by null signal. Each point of the profile represents the distance between the PUH and the local position of the reflective surface. Any cantilever deflection would then results in a change in this defocus distance.

The average of these points gives information about the absolute distance between the PUH and the cantilever (illustrated in Bending, Analysis 1 in FIG. 10), while the profile gives information about the surface properties and its roughness (Surface reconstruction, Analysis 2 in FIG. 10).

Another interesting feature of the astigmatic detection system is the capability of measuring small oscillations of the laser intensity illuminating the PDIC, and analyzes them in the frequency domain. Through FFT processing it is hence possible to determine the resonance frequency of vibrating surfaces measuring the periodic oscillations of the focus error signal they generate. The high resolution of the optical head is able to detect oscillation in the sub-nanometer level, allowing the measuring of cantilevers' vibrational frequencies even in absence of external actuation (Thermal noise, Analysis 3 in FIG. 10). The system was then design considering the simultaneous application of the above mentioned measurement techniques. In our technology we can implement in the same device the simultaneous running of the three analysis: Bending (Analysis 1), Surface reconstruction (Analysis 2) Thermal noise (Analysis 3).

An approach for wobbling compensation was developed modifying the rotating stage and including a mechanical bearing with high-precision rotational properties. Using this approach we implement the calibration methods and the optical modification explained in the previous sections into the same, high-throughput and high-resolution readout device.

In order to eliminate the main source of wobbling (the motor shaft and the clamping metal head), a new approach was implemented. A smaller motor was connected to a high-precision rotating bearing through a pulley belt. The bearing has steel spheres that allow the structure to float over the spheres themselves. The wobbling of the stage thus relies on the precision of the dimensions of the spheres (deviation less than 5 μm, from datasheet).

The rotating bearing and the motor are mounted over an alumium support. Two X-Z linear stages hold the PUHs under the rotating bearing. FIGS. 1-2 illustrate a CAD model of the complete system.

New belt-pulling system: the rotating stage is now composed by a big high-precision ring bearing that is pulled by a belt. This design allows the rotating stage precision to rely on the bearing, instead of on the motor shaft. The bearing has X-Y plane precision of about 5 micron (from datasheet specs). Wobbling is thus in this way highly reduced.

The motor is considerably small. However the belt system magnifies the resolution of a factor equal to the ratio of the two radii (20 times in one embodiment). The small motor has resolution of 50.000 step/revolution (0.072 degrees) that become around 100.000 (0.0036 degrees) after pulley-belt conversion.

As described earlier FIG. 1 is a schematic illustration of parts of a system according to the present invention. The system comprises a device supported on a rotation unit configured or adapted to rotate the device. The device has a substantially disk-shaped geometry, i.e. round and substantially the same width. The device comprises a central opening adapted to engage the rotation device so as to transfer rotational motion to the device. The device comprises a number of fluid channels having an inlet near the central opening, i.e. near the centre of the device. A number of chambers are formed in the fluid channel, here is illustrated three chambers or cavities each comprising a single sensor. The chambers or cavities are in fluid communication with a neighbouring chamber so that fluid may flow from the inlet to the end of the channel. When the device is spun the fluid inputted at the inlet will be forced through the channel due to the centrifugal force arising from the rotation of the device.

The test apparatus or system comprises four optical sensing devices, here indicated as DVD-ROM pickup head.

The sensors in the device is illustrated as silicon cantilevers having a gold coating.

Also, the fluid channel is illustrated as having capillary valves. This is not a requirement for the device to work, but illustrative of an option for the device.

FIG. 2 is a schematic illustration of a system and measurements. The DVD-ROM laser in FIG. 2A detects the properties as mentioned and while the device is spun, the laser scans, as illustrated by the line in FIG. 2B, the sensors. FIG. 2C illustrates measurements of the bending of the cantilevers and FIG. 2D illustrates a zoomed view of the measurements. FIG. 2E illustrates the statistical distribution of the measurements and FIG. 2F illustrates 3D reconstruction of the cantilevers based on the measurements.

FIGS. 3 and 4 are schematic illustrations of sets of measurement results as described elsewhere in the present description.

FIG. 5 is a schematic illustration of a device according to the present invention.

The individual components of an embodiment of a system are indicated. The system is in wireless communication with a computer device acting as output unit. The computer may record and store information from the test system.

FIG. 6A and 6B are schematic illustrations of block diagrams of systems according to the present invention.

FIG. 7 is a schematic illustration of the principle of astigmatism.

FIG. 8 is a schematic illustration of a sensor having 8 cantilevers. One or more of the cantilevers may be used for calibration or reference.

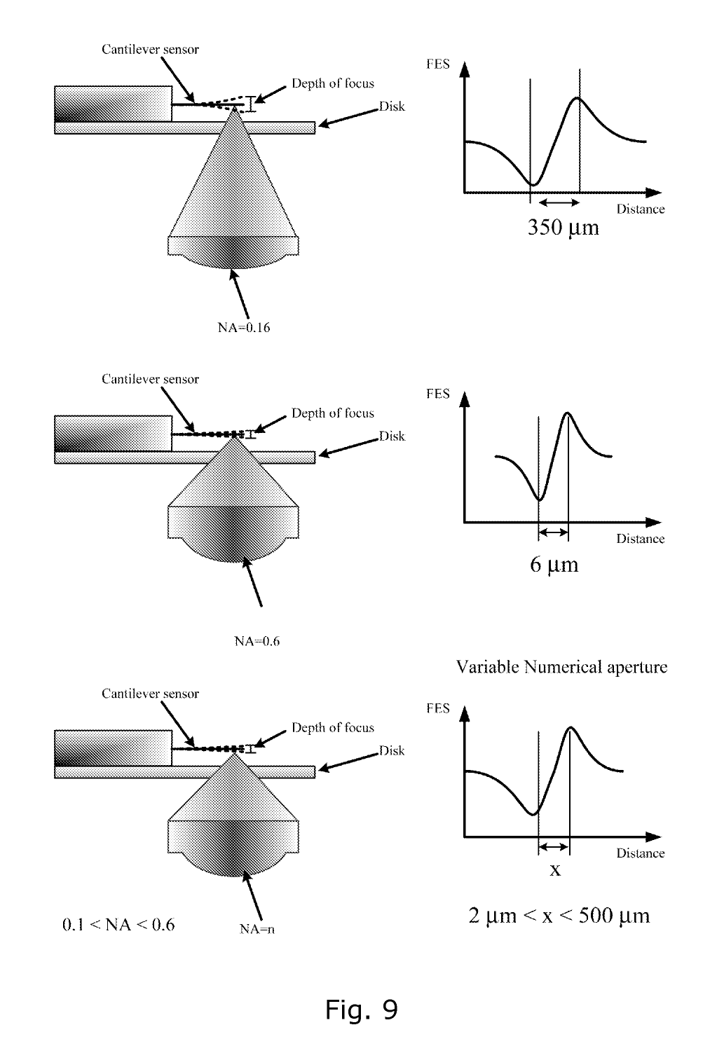

FIG. 9 is a schematic illustration of optical sensors having different numerical apertures. In the upper illustration an optical detector or optical sensing device have a numerical aperture of 0.16, this provides a depth of focus in the range 350 μm as illustrated. In the middle is illustrated an optical detector or optical sensing device have a numerical aperture of 0.6, this provides a depth of focus in the range 6 μm as illustrated. The bottom illustration indicates that a numerical aperture may be chosen in the range 0.1 to 0.6 whereby a depth of focus in the range 2 μm to 500 μm may be achieved.

FIG. 10 is a schematic illustration of different measurements, where the upper and lower left figures illustrates detection of the bending of the individual cantilevers, the middle two figures illustrate measurements for the purpose of data reconstruction of the surface of the cantilevers. The upper and lower left illustrations illustrate measurement for determining the resonant frequency of the cantilevers.

FIG. 11 is a schematic illustration of a device according to the present invention. As illustrates the device comprises three layers, where the top substrate includes the fluid channels. The middle layer is configured to hold the top and bottom substrate together. The bottom substrate includes alignment points for ensuring that the top and bottom substrates are aligned correctly when assembled. The bottom substrate includes a number of SERS chips for performing optical measurements of the Raman scattering.

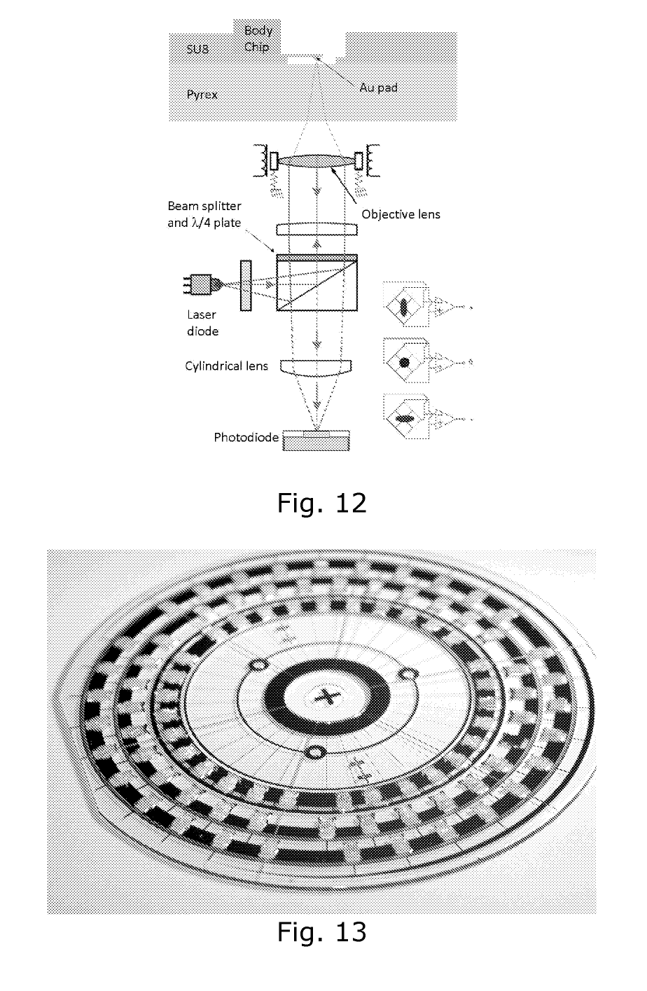

FIG. 12 is a schematic illustration of details of a measurement setup. In the setup a part of the device is illustrated. The device includes a Pyrex body supporting the SU8 and Body chip having an Au pad. Below the device is illustrated the optical device comprising an objective lens, a beam splitter and A/4 plate, a laser diode, a cylindrical lens and a photodiode. The photodiode is also illustrated on the side, where four detectors are used for evaluation of the optical signal.

FIG. 13 is a photograph of a disk for a test system according to the present invention.

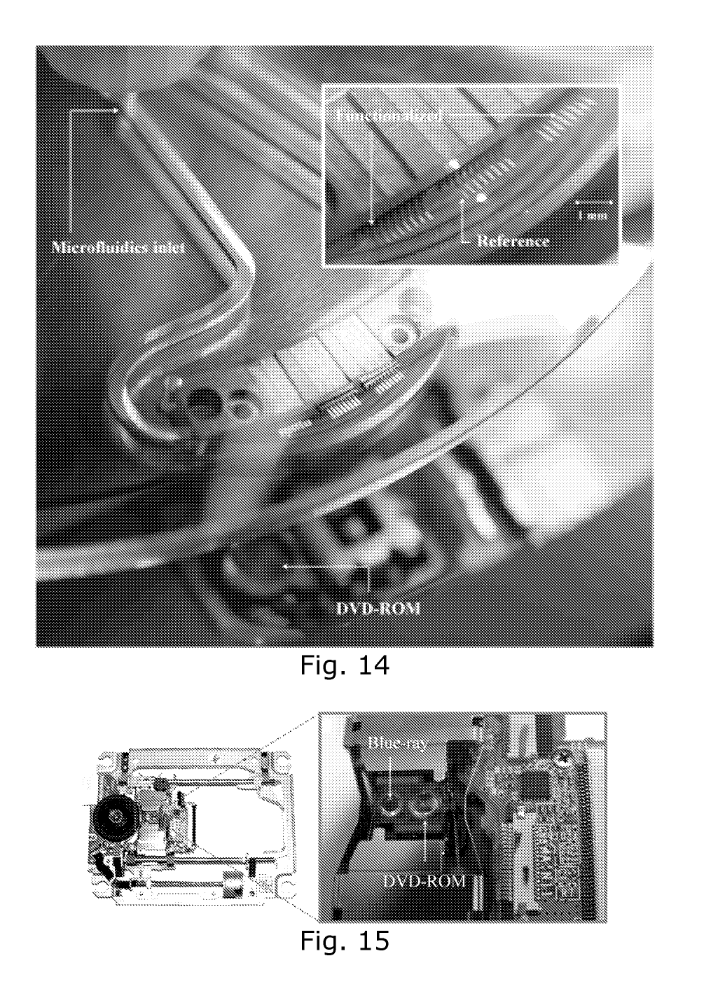

FIG. 14 is a photograph of a part of a device and an optical pick-up head. The sensors have been functionalised by coating them with an appropriate coating.

As also illustrated the fluid channel need not be a straight line from the inlet to the sensors.

FIG. 15 is an image of an optical sensing device having two optical pick-up heads. The unit is taken from a commercially available Blu-ray unit and includes two optical units, one originally used for reading Blu-ray disk, and one originally used for reading DVD-ROM disks.

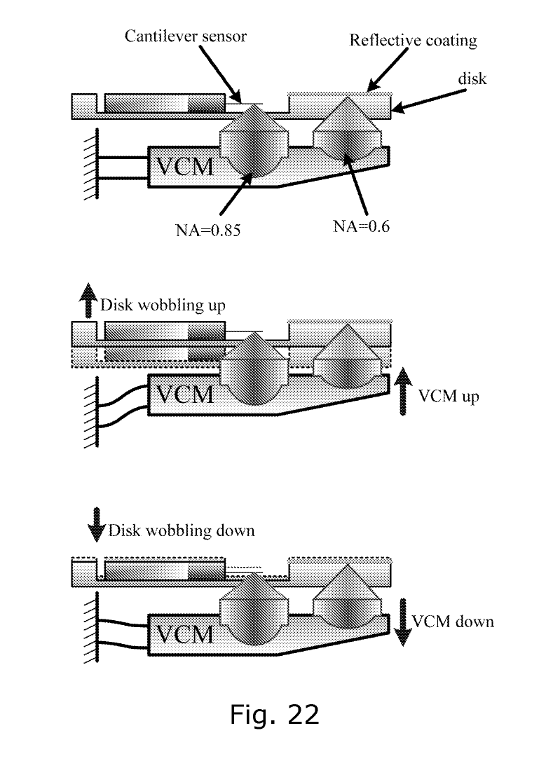

As is illustrated in FIG. 22 the two optical units may be used for other purposes, e.g. one unit may be used for calibration and the other used for detecting or reading the sensors in the device.

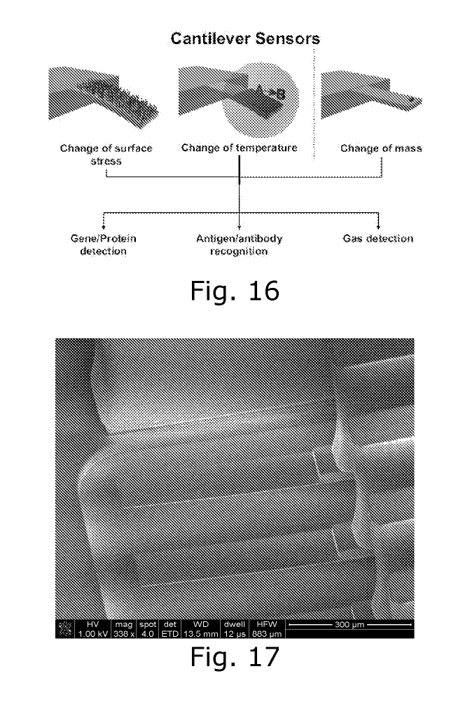

FIG. 16 is a schematic illustration of a cantilever sensor. The presence of a compound or other substance, e.g. virus or other biological matter, will change the properties of the sensor, here illustrated as change of surface stress, i.e. bending of the cantilever. A change in temperature may also change the properties of the cantilever/sensor. Also change in mass of the sensor/cantilever may be detected. These changes may be individually analysed or combined to determine if a substance is present or even the amount/concentration may be determined.

FIG. 17 is an image of a part of a sensor having multiple beams, here only part of the beams is shown.

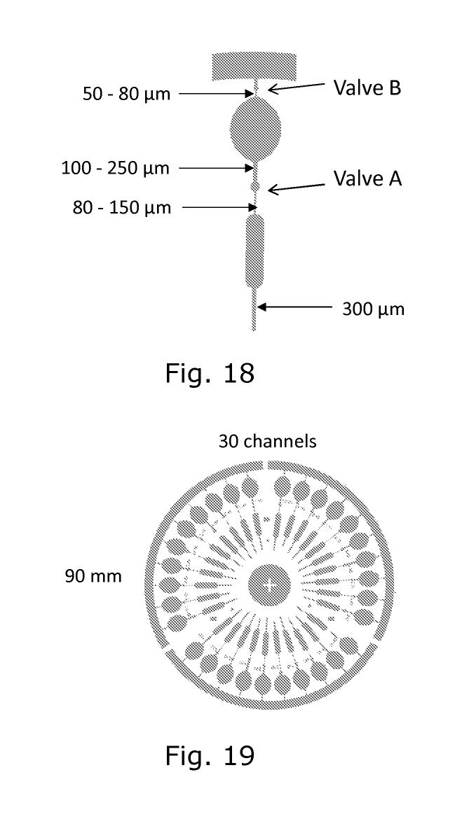

FIG. 18 is a schematic illustration of a fluid channel. An indication of exemplary sizes is given to the individual parts, but other dimension may be applied in other embodiments.

FIG. 19 is a schematic illustration of a device having a number of fluid channels.

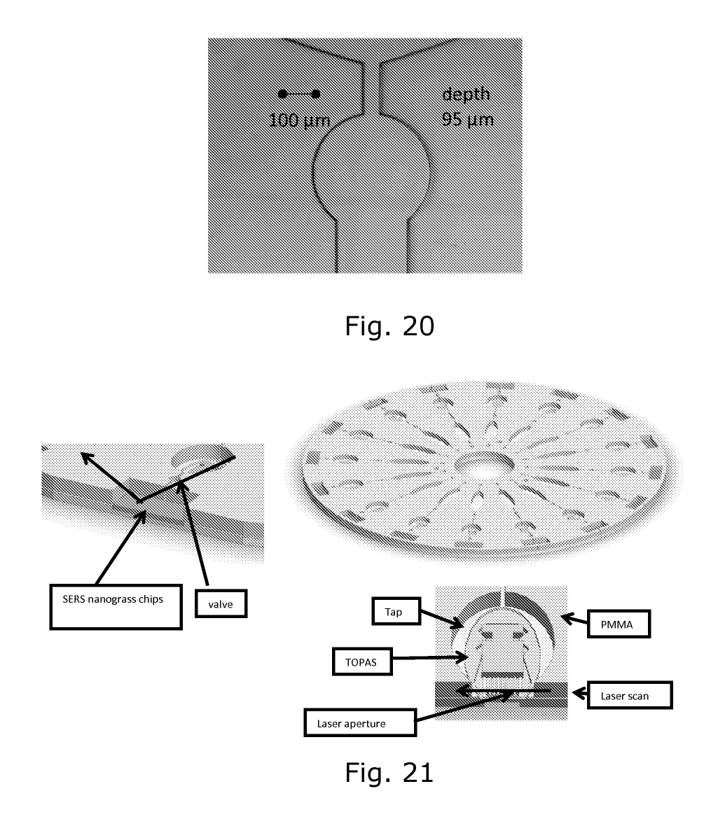

FIG. 20 is an image of a part of a fluid channel.

FIG. 21 is a schematic illustration of a device and close-up illustrations of parts of the device. The measurements of the sensor, i.e. the laser scan, and the measurement of the SERS sensor may be combined or performed individually.

FIG. 22 illustrate the use of two optical pickup heads for calibration and detection of the sensors in the device. The figure illustrates what happens if the device wobbles when being rotated. If the disk wobbles the carrier for the optical units are moved up or down in response to the wobbling so as to ensure that the measurements are performed best possible.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1. An apparatus for detecting compounds, comprising:
a device defining a disk-shaped geometry, the device having a centre, a plurality of fluid channels each comprising a fluid inlet positioned at a first distance from the centre and a fluid channel end at a second distance from the centre, the second distance being larger than the first distance, one or more sensors arranged at each fluid channel, wherein the sensors each comprise at least one optical detectable member,
the apparatus further comprising one or more optical sensing devices arranged for sensing the at least one optical detectable member of the one or more sensors, and
a rotation device adapted for rotating the device so that the sensors pass over the one or more optical sensing devices, wherein the least one optical detectable member is a doubly clamped beam.

2-16. (canceled)

17. The apparatus according to claim 1, wherein two or more sensors are arranged in a fluid channel at different radii.

18. The apparatus according to claim 1, wherein the at least one optical detectable member is a cantilever, a beam, a cantilever beam, or a SERS substrate or any combination thereof.

19. The apparatus according to claim 1, wherein the optical sensing device is arranged so as to sense deflection property, surface property and/or frequency property of the at least one optical detectable member.

20. The apparatus according to claim 1, wherein each of the sensors are arranged in a test chamber in fluid communication with a respective fluid channel.

21. The apparatus according to claim 1, wherein at least one of the optical sensing devices is arranged for detecting wobbling of the device and a controller for determining corrective values for the optical sensing device.

22. The apparatus according to claim 1, wherein the sensor includes receptors, DNA strands, antibodies, antigens or enzymes, which will selectively attract and bond with the particular substance to be detected.

23. The apparatus according to claim 1, wherein at least one of the optical sensing devices has an optical input having a numerical aperture in the interval 0.1 to 0.85.

24. The apparatus according to claim 1, further comprising a connection to a computer device allowing the position of the sensors to be determined and displayed by the computer.

25. The apparatus according to claim 1, wherein at least one of the optical sensing devices includes a first and a second optical receiver, wherein the first optical receiver is adapted for determining wobbling of the device and the second optical receiver is adapted for determining properties of the sensors.

26. The apparatus according to claim 24, wherein the device comprises a patterned ring and the first optical receiver is adapted for calibration by detecting the patterned ring.

27. The apparatus according to claim 1, wherein the sensors are read using astigmatism and the apparatus comprises an optical read head from a CD-player, a DVD-player and/or a Blu-ray player.

28. A method for determining compounds comprising:
providing an apparatus for detecting compounds having a device defining a disk-shaped geometry, the device having a centre, a plurality of fluid channels each comprising a fluid inlet positioned at a first distance from the centre and a fluid channel end at a second distance from the centre, the second distance being larger than the first distance, one or more sensors arranged at each fluid channel, wherein the sensors each comprise at least one optical detectable member, and the least one optical detectable member is a doubly clamped beam,
the apparatus further comprising one or more optical sensing devices arranged for sensing the at least one optical detectable member of the one or more sensors, and
a rotation device adapted for rotating the device so that the sensors pass over the one or more optical sensing devices,
the method comprising:
providing a fluid at an inlet near the centre of the device,
rotating the device, and
obtaining properties of the sensors using the optical sensing devices.

29. The method according to claim 28, further comprising:
determining one or more of: deflection, surface roughness, resonant frequency, or thermal noise of at least one beam of at least one of the sensors.

30. The method according to claim 28, wherein the optical sensing device comprises a first and a second optical receiver, wherein the first optical receiver is adapted for determining wobbling of the device and the second optical receiver is adapted for determining properties of the sensors, the method comprising calibrating the optical sensing device using the signal from the first optical receiver.

31. The method according to claim 28, wherein the apparatus and/or device comprises two or more sensors that are arranged in a fluid channel at different radii.

32. An apparatus for detecting compounds, comprising:
a device defining a disk-shaped geometry, the device having a centre, a plurality of fluid channels each comprising a fluid inlet positioned at a first distance from the centre and a fluid channel end at a second distance from the centre, the second distance being larger than the first distance, one or more sensors arranged at each fluid channel, wherein the sensors each comprise at least one optical detectable member,
the apparatus further comprising one or more optical sensing devices arranged for sensing the at least one optical detectable member of the one or more sensors, and
a rotation device adapted for rotating the device so that the sensors pass over the one or more optical sensing devices, wherein the fluid channel includes a capillary valve.

33. The apparatus according to claim 32, wherein the capillary valve bursts at a certain threshold frequency.

34. A method for determining compounds comprising:
providing an apparatus for detecting compounds having a device defining a disk-shaped geometry, the device having a centre, a plurality of fluid channels each comprising a fluid inlet positioned at a first distance from the centre and a fluid channel end at a second distance from the centre, the second distance being larger than the first distance, one or more sensors arranged at each fluid channel, wherein the sensors each comprise at least one optical detectable member,
the test apparatus further comprising one or more optical sensing devices arranged for sensing the at least one optical detectable member of the one or more sensors, and wherein the fluid channel includes a capillary valve,
a rotation device adapted for rotating the device so that the sensors pass over the one or more optical sensing devices,
the method comprising:
providing a fluid at an inlet near the centre of the device,
rotating the device at a certain angular frequency so that the valves are opened when spinning the apparatus at the certain angular frequency, and
obtaining properties of the sensors using the optical sensing devices.

35. An apparatus for detecting compounds, comprising:
a device defining a disk-shaped geometry, the device having a centre, a plurality of fluid channels each comprising a fluid inlet positioned at a first distance from the centre and a fluid channel end at a second distance from the centre, the second distance being larger than the first distance, one or more sensors arranged at each fluid channel, wherein the sensors each comprise at least one optical detectable member,
the apparatus further comprising one or more optical sensing devices arranged for sensing the at least one optical detectable member of the one or more sensors,
a rotation device adapted for rotating the device so that the sensors pass over the one or more optical sensing devices, and
wherein the optical sensing devices determines deflection of the optical detectable member through a Focus Error signal obtained through differentiating intensities on four quadrants composing the optical sensing device.
